SEQUENCE LISTING (25 sequences)

SEQ ID NO: 1 - DNA, 2085 bp, Homo Sapiens
agatctggccattgcactccagcctgggcaacagagaaaaactccatctaaaaaaaaaaaaaaaaaaaaaaaaaaacagagagagagagagagagagagagaaggaaacggaactggggggaggatttgcaaaaatatggttagggatggcacttcagagatgaagccatcctggagtgttacgggcaagggaaatgctggggcaaagccccagaggcaggaataggtttggcctgttgcatgaacagtgggtccagctcctagcaaactgtttattgaatgaaagaagaatgaatgccttgggtctagggttgtgctgggcgctttcttaagttttctttcccgggtacctccccagaactggcatgcaggtattattaaacccattacacaagtgaaactggcccagagacagaaaagtccctggtccaagaccacacaggagtgaggggtggaggaaccctcctcccattgagttctggctttcctatactgaaagccccttcctctcctgcagtaaggtaggtggaaccgctgtcccgccttgttggtgaatgtcgttgctagacttcagacacatacaggctggtctgctgaaaatcagagatgtccacctgcgccctattcgaggtctccggcgtcttctttggcgtcgtctttgccctttcagaagcgtctgcacatttttccaggtgtcatttctccaacttgaacacagggagcgcactgggcacgcgggcacgtggctgtccccaggggcctggcttgggtctcgcccctgggccggggcgcacgcgcgggcgggacatctgggggcgcccacgcgctctgggacgagtgtcgctggccaggcccggactgaggaaaggcgagtgagacactactcgcctggggtgcaaaatttaagggagtgaaaaaaaaaaaaaaagaaagaaaccaaaaccacctcgagtcaccaaaataaacattttaatgcagtattttttaaaaaatcaacaggaatcctccaaagcccactatgaacaaaatagcaaaatggtagagaaaggatctgtgccgctgcgtcgggcctgtggggcgcctccgggggtctgaaactggaggagactcggggctgtagggcgcgcggatctggggcgcgccctcggtcccggcgcgcccagggcctcccgcgcggggcccggcacagggaggcggggaggcgggcggggcggggcgggaccgggcggcacctccctcccctgcaagctttccctccctctcctgggcctctcccgggcgcagagtcccttcctaggccagatccgcgccgccttttcccgcggcccgcacggggcccagctgacgggccgcgttgtttacgggccggagcagccctctctcccgccgcccgcccgccacccgccagcccaggtgcccgcccgccagtcagctagtccgtcggtccgcgcgtccctctgtcccggagcccgcagatcgcgacccagagcgcgcggggccgagagccgagagacagtcccgggcgcagcgcggagctccgggccccgagatcctgggacggggcccgggccgcagcggccggggggtcggggccaccaccgcaagggcctccgctcagtatttgtagctggcgaagccgcgcgcgcccttcccggggctgcctctgggccctccccggcaggggggctgcggcccgcgggtcgcgggcgtggaagagaaggacgcgcggcccccagcgcctcttgggtggccgcctcggagcatgacccccgcgggccagcgccgcgcgctctgatccgaggagaccccgcgctcccgcagccatgggcaccgggggccggcggggagcggcggccgcgccgctgctggtggcggtggccgcgctgctactgggcgccgcgggccacctgtaccccggagagggtgagtctgggggcgcgggcgtgggcggggagcgccgcgatggggagaggaccccacccaagccaaaatcgatcccccgcttgtggactgagaaccctccccaggggcggggggcggtggccaggacggtagctcctgcatcgcgtagggggagcgggaagc

SEQ ID NO: 2 - DNA, 928 bp, Homo Sapiens
tactttacagagaaagctactcatcccggctggctgcagagtttacagggcccgggatgaaaacacagggcccaggtttcctgtccatgaagccggctctgcccctgatccttctgatgcatccaccgtgcgtctgctcacctgtcttgctttctgttcattttctcttgtagtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataattacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaaggtacgccggggatacagggttctaagcagtgtctcgtgccttgttctagaaagcttaaaatgttttatggcttaaaaatgttaaatggtcattaggtaggggccggggaatagtgggtggtggcattcactagcccagggagtggcagacattttctgtaaagactcagatagtagatacttcagattttgcaggccatatgg

SEQ ID NO: 3 - DNA, 639 bp, Homo sapiens
gatccagaattgctgcatatgcagacaggaattggacaaagccatttatttatttatttatttatttatttatttatttatttatttccctctctctctctctctctctccagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccaatacgtcattcacaacaacaagtgcatccctgagtgtccctccgggtacacgatgaattccagcaagtgagttctggatgtgggtctggggggcagccgagaggagaaggaacgtggggttggttgtgacgatgccgcttgttaaaactgtgtgcaaacccagggttaattggctatgagtgaggtctctgctctcagatgctacttttgcaccctgttttggtcctgggcttgggagtgggagttgactacctttttctctaaaggacc

SEQ ID NO: 4 - DNA, 663 bp, Homo Sapiens
ccaacatggtaaccccgtctctactcaaaaatacaaaaattagccaggcacggtggcgggcacctataatcccagctactgtggaggctgaggcaggagaatctcttgaacccagaaggcagaggttgcagtgagctgagatcgcaccactgcactccagcctgggcaacagagcgagactctgtcacacaaacacacacacacacacaaagaaataccatatcaggcagaaagatgcctgagatgtctgaaggaccttggataccgtgacacccccctcccctttctctttctctctctctctgctccgtccttagcttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcagtgagtgtctctgtgtgggcgtcgggggtgcctgttgggctccatgtccctctgagctgtgagcggggaagaaaagcagtgcagaccctgctgcgtgctcctacagcacttttaggatggtcgttcagtggctcccccatggatagaaccatgctgggagtctgcctcaaaacctgaaatgaacagctcagtcttcc

SEQ ID NO: 5 - DNA, 410 bp, Homo Sapien
gggcagaagtatgcttgacccatttaaggaatgctaaggacttcagattgtgttctaagcatgatgagttttgagctgggtatgtccagtcatttgcagcctgagggttatcttctcaccatggagaatcatgagaagattgaaatatgtctatagaaacccactggatattctctcctttccttagacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggtacgtgggcctgattgtgtgtatggcctgagtgctaactaggaagttcgtgtattagaacaacttaaggattttttt

SEQ ID NO: 6 - DNA, 554 bp, Homo Sapiens
ggccatgaaaacttcctcaacttcctctgttatccacattcaacaaatatgtgttgagtatgtgccaagcaagtggagaggattaggcacgtagcactgaacaagatcaactccgagcatggccacaccatcttggagttgtagaagaccagccgttgaatgactagatgtgtgtgttttttccataggaactactccttctatgccttggacaaccagaacctaaggcagctctgggactggagcaaacacaacctcaccatcactcaggggaaactcttcttccactataaccccaaactctgcttgtcagaaatccacaagatggaagaagtttcaggaaccaaggggcgccaggagagaaacgacattgccctgaagaccaatggggaccaggcatcctgtaagtcactggtccccaacctttttggcacgagggaccggtttagtggaagatggtttttccatggactggtggtgggtggggatggtttcagcatgattcaagtgcattacatttactatgcactttattcctattatgattacattgt

SEQ ID NO: 7 - DNA, 592 bp, Homo Sapiens
ttgcgcgggtacagactgcgcttattcagttgactgtctggctgagtcaagtcattggcttacgtgagtgtgagtggccaagttgcaaaactggctcttacctttgaatcttcccccattcatactcagccaggcacatggggaggagacccttaagggaatagcagcatcacctctgccttctcacggtccctccaggaagtgtgggggtcccaggctttggtctgaaactacactgaaatagctcatttttgccttttgttttaacttttccaggtgaaaatgagttacttaaattttcttacattcggacatcttttgacaagatcttgctgagatgggagccgtactggccccccgacttccgagacctcttggggttcatgctgttctacaaagaggcgtaagtagaagagttagagagacgctgaggaggcgagggctggctggctctgtgcttgctacgtttgtgctccaatctgcccctcttgggttcctgtctatctccctcctcctcctggaataaatatcttaggttcctttttacaatctcaccagtcgatggcatgcaaagtcaatagtgtctgctttt

SEQ ID NO: 8 - DNA, 401 bp, Homo Sapiens
cattagattgttgggtgagtaacatgtgaccctatgggatgtaacttcccaggcctcatctgcacggcactcagtgtgacggtcttgtaagggtaactgccttctgctgttttgtcttgaaagcccttatcagaatgtgacggagttcgatgggcaggatgcgtgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccagtgagtgtgtcttgggaatgtgaattc

SEQ ID NO: 9 - DNA, 420 bp, Homo Sapiens
ggtgccctcatgatgtctttaacttgtgtgtcccccgccatcctcccaccagctttctttgcacactgtttctcatgatggacccgtttcctttctccctggcagacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaaggtgagtgcaggcagctgtgctaggatcggtggggtttgcacacgtgtgtctgatgcactttgcttcacctctagggaagcagctatctcttcctgtgtctcagtgtcggaaggcacacacacacactccattctatctcatatgaaa

SEQ ID NO: 10 - DNA, 517 bp, Homo Sapiens
tttgtggtgtgtgtatgtgtggtgtgttgtgtgatgtgtgtggtgtgtgtgtgggggggtgtgtggtgtgtgtatgtgtggtgtgtgtggtgtgtgtgtgtggtgtgtgtgtgtggggggggtgtgtgtgtgtatgtgtgttcagccgcagagacttgagccccccttttctgtttctttctccagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccaggtcaggacttggcgctgggctctcttagtgggtgccaattggcttggtgttggtggaaggtcattacttagggaccgagaggtagtgggagggagagacggcagaaccctgggtggagtctgaatggag

SEQ ID NO: 11 - DNA, 343 bp, Homo Sapien
tggtccagggtcaaagccagggtgcccttactcggacacatgtggcctccaagtgtcagagcccagtggtctgtctaatgaagttccctctgtcctcaaaggcgttggttttgtttccacagaaaaacctcttcaggcactggtgccgaggaccctaggtatgactcacctgtgcgacccctggtgcctgctccgcgcagggccggcggcgtgccaggcagatgcctcggagaacccaggggtttctctggctttttgcatgcggcgggcagctgtgctggagagcagatgcttcaccaattcagaaatccaatgccttcactctgaaatgaaatctgggcat

SEQ ID NO: 12 - DNA, 719 bp, Homo Sapien
ggtcattcctggcagtctgtattgtaatccatgttccccattgctgcaccctcctgcgctctgatctttcttcttaatcaagccttttattctccagtgtcacttttttaaaaaaaatgatggtgatggtgtcatcatacatgtcctactgtcgttccaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaaggtagggctgctggtccggggtccgagtgtcatgggtgggacatcaaggctgactttttgtttgagacggagccttgctctgtcgcccaggctggagtacagtggtgcgacctcagctcactccagcctctgccacctatgtcaagtgattccctgcttcagcctcccaagtagctgggactacaggtgtctgccaccacgcccagctaatttttgtatttttagtagagatggggtttcaccatattgcccaggctggtcttgaactcctgggctcaagtgatc

SEQ ID NO: 13 - DNA, 439 bp, Homo Sapien
gtcaccagcccaaggttgcaccatggacaggtggcagaagtgggatctcatccaagagttacatccctgcctctcacttcctctccttacagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggtaaggcccttgactcttgggcatgcccctgcaccacttcagcatgccccttcagagttgcacttggtacctccttcctctgctgaaattttgattccagtgcttctctcatcaggtactgtgctattagtacttaaagccttgatacctgacttcgcaggaagatgggtcagaaatgccaatctaccagcttgttacttttcttag

SEQ ID NO: 14 - DNA, 386 bp, Homo Sapien
tggctgtgagctccctgcgaggggtggacactcccagatgtgcaaagctcagccaccctccttctcctcctctcttcctcccaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactattgtaagtctccatggcagcctcagctgactggggctgtgcttagcactgagcatggtgggacattgcaggggatgacttggagaggccgcagtgctggccctggccttgactctcaggcctatcagctgctgcggtgcttgcc

SEQ ID NO: 15 - DNA, 429 bp, Homo Sapien
cccacccattccaggagtggatgtgatttttgatgtgaactttgttggaaacacattgatatgaaacatatattttcttattctatttcagtagacgtcccgtcaaatattgcaaaaattatcatcggccccctcatctttgtctttctcttcagtgttgtgattggaagtatttatctattcctgagaaagaggtgagttcagtgagttcagtggtgtgctgggaacagttggttctctgggggaaaacatgccttgatataggtataggcatatttaagtttattatgaattttgctgatataggatgtgtaacatgcaatttacagataattgtcataatatgatatacacaactctttattgtaaattccctctagacagttgattctcacagaatgtttttattgatttttttttttgcccaaa

SEQ ID NO: 16 - DNA, 480 bp, Homo Sapien
aaaaacaaaaacaaaaacaaaacaaaaaaaaaaccacccagggagggatgagtgctcccatgttgatgcacttacatacctgtctgatgggcttccattcaaaacataaaggtcccccatccctgccctagactgcatctaggattatggggattctgctggtaagggctgccatttgccttggggagtcttgtatgaaacacctttctgcagagtcccatgagaatctcaagctaacgtgcctcgttttcctcctccaggcagccagatgggccgctgggaccgctttacgcttcttcaaaccctgagtatctcagtgccagtgatggtgagtaccatcccttccctgtgggtggccagaaccctactcatcagcttcctttgccttcaccattgagtgagagtgaaggatgggttccccagggaggccaagaaaagccctcttattcatttgagcttgccaaactgcccttgctgcag

SEQ ID NO: 17 - DNA, 485 bp, Homo Sapiens
cccggcatgggtcctggatcacagaactcatttcatgagtgttttcgagggggtttgggtgagggcttgggtggaaggtggctgcagacccccaagggatcctccaaggatgctgtgtagataagtaagaagtagtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcacgtggtgagtccagtgggggtgggacatgggctggctttcctgacccttccctttctctgcctcctcctcctgcacagagcgacagaggacacagggtgtatcctccta

SEQ ID NO: 18 - DNA, 287 bp, Homo Sapien
acgctgcatccaggccacagggtgctgtgtgtgacatagacaccagggagggaggagaaccctggtgagtcgaatcacggaccctcctccaagaaccctggttgcttgctctgcaggtgcgcctcctgggagtggtgtccaagggccagcccacgctggtggtgatggagctgatggctcacggagacctgaagagctacctccgttctctgcggccagaggctgaggtaagctgcttcgggggacccagcggggtactcggtggagcacccgctcctggcctcctc

SEQ ID NO: 19 - DNA, 322 bp, Homo Sapien
gatcccagtgctgctgaaacaccaaccccgtgtttctgttttagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggaggttcgtctggctttctgctttgaaaacataacgacccaggccaggtttgatttcagaaggaagttgtctataatgagccgttaagtcttttctgataatataaaggggcaagtacttc

SEQ ID NO: 20 - DNA, 288 bp, Homo Sapiens
gacgtgggccaggtgaacccctcttagggctctgtgagaggtggggcagtcaaggtggcagatgctaggaccaaggctgaaggttaagagcgtgtgaaccttttgtgttgtcagactttggaatgaccagagacatctatgaaacggattactaccggaaagggggcaagggtctgctccctgtacggtggatggcaccggagtccctgaaggatggggtcttcaccacttcttctgacatgtggtgagttgtgtgtggatgggtggatggacgctgggcttgaattc

SEQ ID NO: 21 - DNA, 407 bp, Homo Sapiens
ttgcgtgtgtgtgtgcgtttgcgtgtgtgtgtttgcgcgcgcgcgtgtgtgtgtgtgtctaaatggcttctttgttactactatcaactgtcatcggcaggtcctttggcgtggtcctttgggaaatcaccagcttggcagaacagccttaccaaggcctgtctaatgaacaggtgttgaaatttgtcatggatggagggtatctggatcaacccgacaactgtccagagagagtgtaagtgtagaaagggtttaaggtgtgtgaggtgttcgttgaaagggtattgccctttacacgtgtgcttggttttgcctttcctatgtctacacgctcaccgtgtttgcatgctgtatgttacaggtgtgtttgtgtttgcatagcttgtctttacatgcatgcttgcatt

SEQ ID NO: 22 - DNA, 873 bp, Homo Sapiens
ctgcagggacaagagtgggggtttgggaggatgcgtggcagggcccccagactcacccaggacgtgtccttctgccccgcagcactgacctcatgcgcatgtgctggcaattcaaccccaagatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaacagtgcctaccgtggcgggggcgggcaggggttcccattttcgctttcctctggtttgaaagcctctggaaaactcaggattctcacgactctaccatgtccagtggagttcagagatcgttcctatacatttctgttcatcttaaggtggactcgtttggttaccaatttaactagtcctgcagaggatttaactgtgaacctggagggcaaggggtttccacagttgctgctcctttggggcaacgacggtttcaaaccaggattttgtgttttttcgttccccccacccgcccccagcagatggaaagaaagcacctgtttttacaaattcttttttttttttttttttttttttttttgctggtgtctgagcttcagtataaaagacaaaacttcctgtttgtggaacaaaatttcgaaagaaaaaaccaaa

SEQ ID NO: 23 - DNA, 4723 bp, Homo Sapiens
ggggggctgcgcggccgggtcggtgcgcacacgagaaggacgcgcggcccccagcgctcttgggggccgcctcggagcatgacccccgcgggccagcgccgcgcgcctgatccgaggagaccccgcgctcccgcagccatgggcaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataatcacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccaatacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccaccactcaggggaaactcttcttccactataaccccaaactctgcttgtcagaaatccacaagatggaagaagtttcaggaaccaaggggcgccaggagagaaacgacattgccctgaagaccaatggggacaaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgatgggcaggatgcgtgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccagaaaaacctcttcaggcactggtgccgaggaccctaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaaattatcatcggccccctcatctttgtctttctcttcagtgttgtgattggaagtatttatctattcctgagaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcacgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaagatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaacagtgcctaccgtggcgggggcgggcaggggttcccattttcgctttcctctggtttgaaagcctctggaaaactcaggattctcacgactctaccatgtccagtggagttcagagatcgttcctatacatttctgttcatcttaaggtggactcgtttggttaccaatttaactagtcctgcagaggatttaactgtgaacctggagggcaaggggtttccacagttgctgctcctttggggcaacgacggtttcaaaccaggattttgtgttttttcgttccccccacccgcccccagcagatggaaagaaagcacctgtttttacaaattcttttttttttttttttttttttttttttgctggtgtctgagcttcagtataaaagacaaaacttcctgtttgtggaacaaaatttcgaaagaaaaaaccaaa

SEQ ID NO: 24 - DNA, 5180 bp, Homo Sapiens
accgggagcgcgcgctctgatccgaggagaccccgcgctcccgcagccatgggcaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataattacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccagtacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccatcactcaggggaaactcttcttccactataaccccaaactctgcttgtcagaaatccacaagatggaagaagtttcaggaaccaaggggcgccaggagagaaacgacattgccctgaagaccaatggggaccaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgacgggcaggatgcatgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaaattatcatcggccccctcatctttgtctttctcttcagtgttgtgattggaagtatttatctattcctgagaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcacgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaacatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaacagtgcctaccgtggcgggggcgggcaggggttcccattttcgctttcctctggtttgaaagcctctggaaaactcaggattctcacgactctaccatgtccaatggagttcagagatcgttcctatacatttctgttcatcttaaggtggactcgtttggttaccaatttaactagtcctgcagaggatttaactgtgaacctggagggcaaggggtttccacagttgctgctcctttggggcaacgacggtttcaaaccaggattttgtgttttttcgttccccccacccgcccccagcagatggaaagaaagcacctgtttttacaaattctttttttttttttttttttttgctggtgtctgagcttcagtataaaagacaaaacttcctgtttgtggaacaaaagttcgaaagaaaaaacaaaacaaaaacacccagccctgttccaggagaatttcaagttttacaggttgagcttcaagatggtttttttggttttttttttttctctcatccaggctgaaggattttttttttctttacaaaatgagttcctcaaattgaccaatagctgctgctttcatattttggataagggtctgtggtcccggcgtgtgctcacgtgtgtatgcacgtgtgtgtgtccattagacacggctgacgtgtgtgcaaagtatccatgcggagttgatgctttgggaattggctcatgaaggttcttctcaagggtgcgagctcatccccctctctccttccttcttattgactgggagactgtgctctcgacagattcttcttgtgtcagaagtctagcctcaggtttctaccctcccttcacattggtggccaagggaggagcatttcatttggagtgattatgaatcttttcaagaccaaaccaagctaggacattaaaaaaaaaaaaaagaaaaagaaagaaaaaacaaaatggaaaaaggaaaaaaaaaaagaactgagatgacagagttttgagaatatatttgtaccatattta

SEQ ID NO: 25 - DNA, 7240 bp, Homo Sapien
gagctccctgcgaggggtggacactcccagatgtgcaaagctcagccaccctccttctcctcctctcttcctcccaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactattgtaagtctccatggcagcctcagctgactggggctgtgcttagcactgagcatggtgggacattgcaggggatgacttggagaggccacaggtgctggccctggccttgactctcaggcctatcagctgctgcggtgcttgccctctttgatcctgcactttttttttttttgagatggaggcttgctttggagtgcactggcacaatctcagctcactgtagcctccgcctcccgggttcaagtgattctcccacctcagcctcacagtagctgggactacaggtgcccaccaccacgcccggctaattcttgtatttttagtagagatggcatttcaccatgttggccaggctggtctcaaactcctgacctcaagtgatccgcccacctcggcctcccaaagtgctggaattacaggcatgagccaccatgcctggcctgatcctgcacttaaaaaaaaaaaaaaaaaaagtttcagaggtactcgtgcagttcattatataagtaaattgtggctgggcacggtggctcacacctgtaatcccagcactttgggaggccgaggcgggcagatcacaaggtcaggagatcgagaccatcctggccaacatggtgaaaccccatctctactaaaaatacaaaaataaattagccaggcatggtggcgggcgcctgtagtcccagctactcaggaggctgaggcaggagcctcaggaacccgggaagcagagcttgcagtgaaccgagatcgtgccactgcactccagcctgggcaacacagtgagactccttctcaaaaaataaaataaaataagtaaattgggtgttgttgggggtttgctgtacagataattttgtcacccatgtaatcagcatagtacctgataggtcgttttttgatcctttccctcttctcacccaccactctcaagtaggcacctgtgttagtctgtacttacactgcaataaagaaatacctggccgggcacagtggctcacacctgtaatcccagcactttgggaggccgaggtgggcggatcacttgaggtcaggagttcgagaccagcctgaccaacatggtgaaaccccatctctacccaaaaatacaaaaattagctgggcatagtggtgtgcacccgcagtcccagctactcaggaggctgaggcaggagaatcacttgaacccgggaggcggaggttgcagtgagccgacatcacgccactgcactccagcctgggtgacagagtgagactctgtctcaaaaaataaaaaagaaagaaagagtgaaagagagagagaagaaaaagaaaaagaaagaaaaggaaagaaagagagaaaaagagagataaaagaaagaaaaagaaagaagagaagagagaggaagaggaagaggtatgcgactgggactcagtaatttataaagaaaagaggtttaattggctcccagatctgcaggcagtacaggaaccatgatgctggcatctgctcagcttctgaggaagcctcaagaaactttcaatcatggtggaaggtgaagtgggagcaaggtgttaagacggggagatggtgctacacacttcttaaacaaccagatcccatgagaacccacttattatacagtacccagtagggatgttgctaacccattagaaaccgcctccatgatccaatcacctcccacccggccactcctccaacattggggattacatttcaacaagagctttgggtggggacacagatccaaaccatagcagtcccggtgtctactgttcacttctttctgtccatgtgtggtcagtgtctcactctcacttatgggtgagaacatgaggtagttggttttctgtccctgtgttaattcaattaggataatcatctccagctccattcatgttgctgcaaagaacatgatctcattctttttcatggctgtgtagtattccatggtgtgtatgtataacattttctttatctggcaatcctgcacttcctcatctgtacatggagataataacagaaccacttcaggaggtggagggacattttaatgacacaaatgttaagtgcctggcacctgttgctagtgtctccatctttgttactagagttttttggctagatgaggtggctcacacctgtaatcccagaactttgggaggctgaggcaacaggattgcttgaggacaggcattagagaccagcctgagcaacatagcgagactctgtctccacaaaaaaatacaaaaattagccagctatggtggtgcatgcttgtaatcccaggtacttgggaggctgagacagggggatcacttgggcccaggaatttgaggttgcggtgaactgtgattgtgccactatactccagcctgggtgacagagtaagaccctgtctctaaaaaataaaaattaaaagaagtttacatctgtcaaaagtcatgctgggatcgggactagctatgtaatttgcaagacccagtgaacaatgaaaatgcagaactccttgctttaaaattattaagaatttggccgggcacgttggctcacgcctgtcatcccagtactttcggaggctgaggcgggaggatcacctgaggtcaggagtttgaggccagcctggccagcaaggtgaaaccctgtctctattaaaaatacaaaaattagccgggtgtggtggtgcatgcctgtagtcccagctacttgggaggctgaggcaggagaatcacttgaacccgaaaggaggaggttccagtgagccgagatcgtgccactgaactctagcctgggtgtcagagcaagactctgtcacaaaaataagtaaataaataaaaattaaaataaaatgaataagcatttcagaggggcaacagcagagcattaaactgacagaaaagggtcctgcatccactgcctgagatgtgggagggatggaaatgagcagtgatttggggcaggggtggggaagagtgtgcttccagaatactgacctctgagcccactgcctggtcccactgcacctacgggactgtttcgggactgctggaaaatcaggatgtggaagagcagcagagaggttcatggacaagggagggaaggaacagggtggcccacccattccaggagtggatgtgatttttgatgtgaactttgttggaaacacattgatatgaaacatatattttcttattctatttcagtagacgtcccgtcaaatattgcaaaaattatcatcggccccctcatctttgtctttctcttcagtgttgtgattggaagtatttatctattcctgagaaagaggtgagttcagtgagttcagtggtgtgctgggaacagttggttctctgggggaaaacatgccttgatataggtataggcatatttaagtttattatgaattttgctgatataggatgtgtaacatgcaatttacagataattgtcataatatgatatacacaactctttattgtaaattccctctagacagttgattctcacagaatgtttttattgatttttttttttgcccaaacctttatatccgaagctaacctattattgcaattgataaacaagtaaagctccaatgtgaatgttgattaatttttcaaaatttacattaaggagtaggacttgactgggcacagtggctcacacctgtaatgctagcactttgggaggccaaggcgggtgaatcacctgaggtcaggagtttgagaccagcctggccaacatggtgaaacctcgtctctactaaaaatactaaaaaattaaccgggcatggtggtgggcgcctgtaatcccagctactcaggaggctgaggcaggagaattactgaaccctggtggcggaggttgcagtgagctgaaatcgcaccattgcattctagcctgggcgacagagggagactccgtctcgggaaaaaaaaaaaaagtaggacaaaactgaaataagacatatatgttcatcagtgatatgagtgacgtctttgctgagtcagatggtaatttttaaatatcagaagaacattttgtgccacatgcaacatcacagttgcagacatgacacgcttttaagtttaatctacatgattaaacatttttctcagctgggcacggtggctcacacctgtaatcccaacactttgggaggccgaggcgggcggatcatgaggtcaggagttcgagaccagcctgaccaacatggtgaaaccccgtctctactaaaaatacaaaaattagcaaggtgtggtgatgtgcgcctgtaatcccagcttctcaggaggctgaggcaggagaatcacttgaacccaggaggcagaggttgcagtgagccgagatcgcaccattgcactccagcctgggcaacagagtgagactttgtctcaaaaacaaaacaaaacaaaaaaatatttttctcatcactttctcaagcctggacaaacaacagaacaacaaatccagtcctgagttatagcatttgccagtttctgtaatgtaaatattcccaggatgtctaaattcaagctgtagacataatattactgagtgcagtgttagaaagagatacataatagctccccattgaatccaccctatggatacaatatggtgtataaatgatataatgtaaataacctcaactgcattgatcatatttaaatgtagtatgagagttaggaagtgatgagttttgaacatgtattgtctttgcttttaggataatttatttaattgtaagcctctataatttatattttttgttctatttggaaggcattgtaaaatttaatctttaatgatgcttgtatttaacaactggctcactagtttcctgaaaatttaataattgtttctcatcagtcgggatgagctcgctctagaacagtactgggtgagtggcttttaagtgttacatggatggccataaattatttaaaaagccagccagagccctgcatggtcgtgcatatctgtagtcccagccgctcgggaggatgaggcaggaggatcacttgagaccaagagttcaagaccagcctgggcaacatagtgagaccctgtctctatgaaattttacaaattagccaggtgtggtggtgagcacctgtattcccagctattcagaaactgaagtgggaggatctctggagcccagaaggttaagactgcagtgagctatgattataccactgccctccagccacaacagagcaagactgcaactctgaaatgtaaaaacaaaaacaaaaacaaaacaaaaaaaaaaccacccagggagggatgagtgctcccatgttgatgcacttacatacctgtctgatgggcttccattcaaaacataaaggtcccccatccctgccctagactgcatctaggattatggggattctgctggtaagggctgccatttgccttggggagtcttgtatgaaacacctttctgcagagtcccatgagaatctcaagctaacgtgcctcgttttcctcctccaggcagccagatgggccgctgggaccgctttacgcttcttcaaaccctgagtatctcagtgccagtgatggtgagtaccatcccttccctgtgggtggccagaaccctactcatcagcttcctttgccttcaccattgagtgagagtgaaggatgggttccccagggaggccaagaaaagccctcttattcatttgagcttgccaaactgcccttgctgcagaaacctcattactgtgtgcatctggacacatggtatttggcacctgcctgaatgggctcatctagccggtctgggacccttgggcagggtcgaccacttgggctgggctcagctgggcggttcttctggtcttgcctggcttcacccatgtagctacattttgctggtgtgtcagctagcactcggtagtcttagatgatttcactcacatgtctggtggtcagcaggctgggtggtcccaaggtgggggccctaagctgggggaggctgagcctcactctgtccatctagcctcttaggctccagcaggctggctcaggcttcattccatggtcctctgttggttcctagtagcaagctccagggcaagctccagggcaacagtccattccaaatctctgcttggacaattcttgttgattcccattgaccaaaataactcacaaggccatgcccagggccaaggggtggtgagatagactccaccttttcatgggaagagctccaagtatcctggcaaaaaaaaaccccaacctattacaacctgtcttccatccccttggcactttgcagaaacagtagtctcaggtgggaagtagcatcattccatagcaagggtctgaaatcagacaagaaggatggggatgcaggtttgcctcaggacatattggccaggatcttggaccagttgtggctccttccttgagtctctgccatgccctctccatgggtgcagatgcctgtcctgttctcggccatatgcccagtgcccggcatgggtcctggatcacagaactcatttcatgagtgttttcgagggggtttgggtgagggcttgggtggaaggtggctgcagacccccaaggatcctccaaggatgctgtgtagataagtaagaagtagtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcatgtggtgagtccagtgggggtgggacacgggctggctttcctgacccttccctttctctgcctcctcctcctgcacagagcgacagaggacacagggtgtaacctcctacccacccctcactccactaagctt

(19) United States
(12) Patent Application Publication
(10) Pub. No.: US 2005/0256649 A1
(43) Pub. Date: Nov. 17, 2005

(54) High throughput correlation of polymorphic forms with multiple phenotypes within clinical populations

(76) Inventor: Allen D. Roses, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/496,711

(22) PCT Filed: Dec. 18, 2002
(86) PCT No.: PCT/US02/40358

§ 371 Date: May 26, 2004

Related U.S. Application Data
(60) Provisional application No. 60/344,892, filed on Dec. 21, 2001.

(51) Int. Cl. G01N 33/48; G06F 19/00

(52) U.S. Cl. 702/19; 702/20

(57) ABSTRACT

A computer-assisted method of looking for pharmacologic targets, in which large numbers of persons are enrolled in drugh clinical trials, they are medically examined and documented, tissue samples are taken, the tissue samples are genotyped, and an examination is made of the genotypes to try to ascertain associations between the genotypes and the documented disease phenotypes of the patients.

### FIELD OF THE INVENTION

The present invention relates to the field of genetics in general and the field of pharmacogenetics in particular, with specific application to the problem of how to derive associations between a given genotype and a given phenotype in a human population exhibiting multiple disease genotypes.

### BACKGROUND OF THE INVENTION

Over recent years, much progress has been made in mapping and sequencing the human genome, to the present point where at least an initial draft of the fill coding sequence of every human gene is known. However, determining the function of these newly identified genes, or conversely, identifying phenotypes associated with genes, has proceeded more slowly. Elucidation of function can be particularly difficult in situations in which a single gene contributes to several phenotypes and/or where multiple genes contribute to a single phenotype. Such situations may prove to be the norm rather than the exception: Existing approaches to correlating genetic polymorphism and phenotype often start by selecting a single phenotype of interest. A population having the phenotype is selected together with a control population who lack the phenotype. DNA is extreacted from both populations and co-segregational linkage between the phenotype and polymorphic markers in the DNA is performed. Usually the analysis initially identifies polymorphic markers spaced some distance from the gene associated with the phenotype. By a variety of approaches, such as direct cloning, it is often possible to identify markers progressively closer to the gene until evnentually the gene itself I identified.

Having found a variant form of a gene, or a polymorphic marker that correlates with a single disease phenotype, the above approach has, in some instances, been extended to look for correlations with one or more additional phenotypes. The approach in identifying a correlation with a second phenotype has been very similar to that for the first phenotype. That is, a further population of individuals is identified that have the second phenotype. This population typically has entirely different individuals from the population having the first phenotype. One then tests for a correlation between the variant gene or polymorophic marker and the population having the second phenotype in comparision with a control population.

The present invention however seeks to maximize the value of having a relatively large population of persons enrolled in clinical trials to test the efficacy and safety of human drugs prior to approval of such drugs for sale in the marketplace. Such persons are characterized as having been extensively examined and evaluated by trained and experienced medical personnel, and thereofore in having a relatively large volume of well detailed and written medical reports and lab test results. Tissues taken from such well-documented patients are then much more valuable for the potential to find correlations between given polymorphisms of genotypes with the well-documented phenotypes in the form of clinical diseases. Unlike all previous approaches to the problem of finding associations between phenotypes and genotypes, the method of the present invention operates by systematically collecting the records and tissue samples of a large number of patients in such trials, genotyping the samples on a high-throughput scale of activity and then using bioinformatic algorithms to find the associations between the genotypes of the samples and the disease phenotypes, so that the end result is the obtaining of a desired number of associations between genotypes and not just one, but multiple disease phenotypes.

### SUMMARY OF THE INVENTION

In summary, the claimed invention is a method of datamining data obtained from a population of humans in clinical trials, across multiple diseases, for associations between said diseases and multiple genotypes, and performed in a programmable digital computer, comprising the steps of:
(a). providing a database having, for each member of a subject population, a first value set specifying at least one polymorphic form selected from a plurality of polymorphic forms present in said population at at least one genetic locus exhibiting polymorphism, and a second value set specifying a plurality of phenotypes, wherein at least one of said polymorphic forms is not known to have a statistically significant correlation with at least one of said phenotypes; and
(b). determining all possible statistical correlations between the plurality of polymorphic forms and the plurality of phenotypes.

### DETAILED DESCRIPTION OF THE PREFERRED EMBODIMENTS OF THE INVENTION

A number of patients is enrolled in a clinical trial. The patients are carefully interviewed, examined and tested in accordance with best medical practice in the area involving a given disease, for example asthma or neurolgy. Tissue samples are taken and genotyped, and the resulting genotypic data is analyzed with the computerized aid of bioinformatic algorithms to establish the presence of an associaiton between the resulting genotype and the known phenotype of the examined patient.

An example of how this is done with a single disease, in this example, migraine headache, is given in the following example.

### EXAMPLE 1

### Association between a Genotype and Susceptibility to Cephalic Pain

The Example relates to the diagnosis of susceptibility to cephalic pain and agents which can be used in the diagnosis of cephalic pain.

Cephalic pain disorders are generally multifactorial disorders, many of which have an unknown etiology. No biochemical marker had been found for many of these disorders, and therefore diagnosis could only be done by clinical symptoms. Both environmental and genetic factors are thought to contribute to cephalic pain disorders. In the case of susceptibility to migraine familial aggregation is observed, and segregation analysis of the pattern of inheritance of migraine within families indicates a multifactorial inheritance (not a simple Mendelian inheritance). A multifactorial inheritance means that many genes contribute to the genetic predisposition to migraine, making it difficult to identify the individual susceptibility genes in linkage studies.

In this Example, it is shown that the insulin receptor is involved in the etiology of migraine. It was found that polymorphisms in the insulin receptor gene cause susceptibility to cephalic pain, and in particular to migraine.

Accordingly, the Example provides a method of diagnosing susceptibility to cephalic pain in an individual comprising typing the insulin receptor gene region or insulin receptor protein of the individual and thereby determining whether the individual is susceptible to cephalic pain.

Description of Sequences in Sequence Listing

SEQ ID NOS: 1 to 22 are the sequences of exons 1 to 22 of the insulin receptor gene;
SEQ ID NO: 23 is the complete coding sequence of the insulin receptor mRNA;
SEQ ID NO: 24 is the sequence of the mRNA for the insulin receptor precursor; and
SEQ ID NO: 25 is the complete sequence from exons 14 to 17 of the insulin receptor gene, including introns.

The insulin receptor gene region or insulin receptor protein of an individual is typed. The individual's susceptibility to cephalic pain can thus be determined. The cephalic pain is typically a cluster headache, chronic paroxysmal hemicrania, headache associated with vascular disorders, headache associated with substances or their withdrawal (for example drug withdrawal), tension headache and, in particular, migraine with aura or migraine without aura.

The typing of the insulin receptor gene region or insulin receptor protein may comprise the measurement of any suitable characteristic of the gene region or receptor to determine whether the individual is susceptible to cephalic pain. Typically the characteristic which is measured is one which can be influenced by a cephalic pain susceptibility polymorphism in the insulin receptor gene region or protein (e.g any such polymorphism mentioned herein). The individual may or may not have a cephalic pain susceptibility polymorphism, but the gene region or receptor may have been affected by other factors (environmental or genetic) which have caused an effect which is similar to the effect of the susceptibility polymorphism. Such an effect may be any of the effects of the polymorphisms discussed herein.

Typically the typing comprises identifying whether the individual has a cephalic pain susceptibility polymorphism, or a polymorphism which is in linkage disequilibrium with such a polymorphism, in (i) the insulin receptor gene region or (ii) the insulin receptor protein.

Polymorphisms

Polymorphisms which are in linkage disequilibrium with each other in a population tend to be found together on the same chromosome. Typically one is found at least 30% of the times, for example at least 40%, 50%, 70% or 90%, of the time the other is found on a particular chromosome in individuals in the population. Thus polymorphisms which are not functional susceptibility polymorphisms, but are in linkage disequilibrium with the functional polymorphisms, may act as a marker indicating the presence of the functional susceptibility polymorphism. Polymorphisms which are in linkage disequilibrium with any of the polymorphisms mentioned herein are typically within 500 kb, preferably within 400 kb, 200 kb, 100 kb, 50 kb, 10 kb, 5 kb or 1 kb of the polymorphism. Similarly the term “insulin receptor gene region” generally encompasses any of these distances from 5′ to the transcription start site and 3′ to the transcription termination site.

As mentioned above the polymorphism which is typed may be in the insulin receptor gene region or protein. The polymorphism is typically an insertion, deletion or substitution with a length of at least 1, 2, 5 or more base pairs or amino acids.

In the case of a gene region polymorphism, the polymorphism is typically a substitution of 1 base pair, i.e. a single polynucleotide polymorphism (SNP). The polymorphism may be 5′ to the coding region, in the coding region, in an intron or 3′ to the coding region. The polymorphism which is detected is typically the functional mutation which contributes to cephalic pain, but may be a polymorphism which is in linkage disequilibrium with the functional mutation.

Thus generally the polymorphism will be associated with cephalic pain, for example as can be determined in a case/control study (e.g. as mentioned below). The polymorphism will generally cause a change in any of the characteristics of the receptor discussed herein, such as expression, activity, expression variant, cellular localisation or the pattern of expression in different tissues. The agent may modulate any of the following activities of the insulin receptor: insulin binding, IGF-1 binding, kinase activity (e.g. tyrosine, threonine or serine kinase activity), autophosphorylation, internalisation, re-cycling, interactions with regulatory proteins, or interactions with signalling complexes. The polymorphism may modulate the ability of the receptor to cause directly (or indirectly through another component) post-translational modifications, such as serine/threonine phosphorylation, dephosphorylation (via serine /threonine- or tyrosine phosphatases) or glycosylation.

The polymorphism typically has an agonist or antagonist effect on any of these characteristics of the receptor. Generally this will lead to a consequent increase or decrease in the activity of the pathway.

In a preferred embodiment the polymorphism causes reduced sensitivity to insulin. Typically such a polymorphism will cause reduced binding of the insulin receptor to insulin.

The polymorphism may be any of the following polymorphisms: INSBa, INSCa, exon8.pol1, exon11.pol1, exon17.pol2, exon6.poll, exon7.pol1, exon7.pol2, exon8.pol2, exon9.pol3, exon14.poll or INSR-c.4479C>T. These polymorphisms are defined in Table 1 below with reference to the sequence flanking the polymorphism. The form of the polymorphisms is allele 2 as defined in Table 1 for each of INSBa, INSCa, exon8.pol1, exon11.pol1 and exon17.pol2. For each of exon6.pol1, exon7.pol1, exon7.pol2, exon8.pol2, exon9.pol3, exon14.pol1 and INSR-c.4479C>T, the form of the polymorphism is allele 1 or 2 as defined in Table1. Each of exon6.pol1, exon7.pol1, exon7.pol2, exon8.pol2, exon9.pol3, exon14.pol1 and INSR-c.4479C>T is in linkage disequilibrium with one of the associated polymorphisms, i.e. with one of INSBa, INSCa, exon8.pol1, exon11.pol1 and exonl7.pol2.

The polymorphism may be a polymorphism at the same location as any of these particular polymorphisms (in the case of a SNP, it will be an A, T, C or G at any of the locations). The polymorphism may be in linkage disequilibrium with any of these particular polymorphisms. The polymorphism will have a sequence which is different from or the same as the corresponding region in any one of SEQ ID NOS: 1 to 25. A polymorphism which can be typed to determine susceptibility to cephalic pain may be identified by a method comprising determining whether a candidate polymorphism in the insulin receptor gene region or insulin receptor protein is (i) associated with cephalic pain or (ii) is in linkage disequilibrium with a polymorphism which is associated with cephalic pain, and thereby determining whether the polymorphism can be typed to determine susceptibility to cephalic pain.

Detection of Polymorphisms

The polymorphism is typically detected by directly determining the presence of the polymorphism sequence in a polynucleotide or protein of the individual. Such a polynucleotide is typically genomic DNA or mRNA, or a polynucleotide derived from these polynucleotides, such as in a library made using polynucleotide from the individual (e.g. a cDNA library). The processing of the polynucleotide or protein before the carrying out of the method is discussed further below.

Typically the presence of the polymorphism is determined in a method that comprises contacting a polynucleotide or protein of the individual with a specific binding agent for the polymorphism and determining whether the agent binds to a polymorphism in the polynucleotide or protein, the binding of the agent to the polymorphism indicating that the individual is susceptible to migraine.

Generally the agent will also bind to flanking nucleotides and amino acids on one or both sides of the polymorphism, for example at least 2, 5, 10, 15 or more flanking nucleotide or amino acids in total or on each side. Generally in the method, determination of the binding of the agent to the polymorphism can be done by determining the binding of the agent to the polynucleotide or protein. However in one embodiment the agent is able to bind the corresponding wild-type sequence by binding the nucleotides or amino acids which flank the polymorphism position, although the manner of binding will be different to the binding of a polynucleotide or protein containing the polymorphism, and this difference will generally be detectable in the method (for example this may occur in sequence specific PCR as discussed below).

In the case where the presence of the polymorphism is being determined in a polynucleotide it may be detected in the double stranded form, but is typically detected in the single stranded form.

The agent may be a polynucleotide (single or double stranded) typically with a length of at least 10 nucleotides, for example at least 15, 20, 30 or more polynucleotides. The agent may be molecule which is structurally related to polynucleotides that comprises units (such as purines or pyrimidines) able to participate in Watson-Crick base pairing. The agent may be a protein, typically with a length of at least 10 amino acids, such as at least 20, 30, 50, 100 or more amino acids. The agent may be an antibody (including a fragment of such an antibody which is capable of binding the polymorphism).

A polynucleotide agent which is used in the method will generally bind to the polymorphism, and flanking sequence, of the polynucleotide of the individual in a sequence specific manner (e.g. hybridise in accordance with Watson-Crick base pairing) and thus typically has a sequence which is fully or partially complementary to the sequence of the polymorphism and flanking region. The partially complementary sequence is homologous to the fully complementary sequence.

In one embodiment of the method the agent is a probe. This may be labelled or may be capable of being labelled indirectly. The detection of the label may be used to detect the presence of the probe on (and hence bound to) the polynucleotide or protein of the individual. The binding of the probe to the polynucleotide or protein may be used to immobilise either the probe or the polynucleotide or protein (and thus to separate it from one composition or solution).

In one embodiment the polynucleotide or protein of the individual is immobilised on a solid support and then contacted with the probe. The presence of the probe immobilised to the solid support (via its binding to the polymorphism) is then detected, either directly by detecting a label on the probe or indirectly by contacting the probe with a moiety that binds the probe. In the case of detecting a polynucleotide polymorphism the solid support is generally made of nitrocellulose or nylon. In the case of a protein polymorphism the method may be based on an ELISA system.

The method may be based on an oligonucleotide ligation assay in which two oligonucleotide probes are used. These probes bind to adjacent areas on the polynucleotide which contains the polymorphism, allowing (after binding) the two probes to be ligated together by an appropriate ligase enzyme. However the two probes will only bind (in a manner which allows ligation) to a polynucleotide that contains the polymorphism, and therefore the detection of the ligated product may be used to determine the presence of the polymorphism.

In one embodiment the probe is used in a heteroduplex analysis based system to detect polynucleotide polymorphisms. In such a system when the probe is bound to polynucleotide sequence containing the polymorphism it forms a heteroduplex at the site where the polymorphism occurs (i.e. it does not form a double strand structure). Such a heteroduplex structure can be detected by the use of an enzyme which is single or double strand specific. Typically the probe is an RNA probe and the enzyme used is RNAse H which cleaves the heteroduplex region, thus allowing the polymorphism to be detected by means of the detection of the cleavage products.

The method may be based on fluorescent chemical cleavage mismatch analysis which is described for example in PCR Methods and Applications 3, 268-71 (1994) and Proc. Natl. Acad. Sci. 85, 4397-4401 (1998).

In one embodiment the polynucleotide agent is able to act as a primer for a PCR reaction only if it binds a polynucleotide containing the polymorphism (i.e. a sequence- or allele-specific PCR system). Thus a PCR product will only be produced if the polymorphism is present in the polynucleotide of the individual. Thus the presence of the polymorphism may be determined by the detection of the PCR product. Preferably the region of the primer which is complementary to the polymorphism is at or near the 3′ end of the primer. In one embodiment of this system the polynucleotide agent will bind to the wild-type sequence but will not act as a primer for a PCR reaction.

The method may be an RFLP based system. This can be used if the presence of the polymorphism in the polynucleotide creates or destroys a restriction site which is recognised by a restriction enzyme. Thus treatment of a polynucleotide with such a polymorphism will lead to different products being produced compared to the corresponding wild-type sequence. Thus the detection of the presence of particular restriction digest products can be used to determine the presence of the polymorphism.

The presence of the polymorphism may be determined based on the change which the presence of the polymorphism makes to the mobility of the polynucleotide or protein during gel electrophoresis. In the case of a polynucleotide single-stranded conformation polymorphism (SSCP) analysis may be used. This measures the mobility of the single stranded polynucleotide on a denaturing gel compared to the corresponding wild-type polynucleotide, the detection of a difference in mobility indicating the presence of the polymorphism. Denaturing gradient gel electrophoresis (DDGE) is a similar system where the polynucleotide is electrophoresed through a gel with a denaturing gradient, a difference in mobility compared to the corresponding wild-type polynucleotide indicating the presence of the polymorphism.

The presence of the polymorphism may be determined using a fluorescent dye and quenching agent-based PCR assay such as the Taqman PCR detection system. This is illustrated in FIG. 1. In brief, this assay uses an allele specific primer comprising the sequence around, and including, the polymorphism. The specific primer is labelled with a fluorescent dye at its 5′ end, a quenching agent at its 3′ end and a 3′ phosphate group preventing the addition of nucleotides to it. Normally the fluorescence of the dye is quenched by the quenching agent present in the same primer. The allele specific primer is used in conjunction with a second primer capable of hybridising to either allele 5′ of the polymorphism.

In the assay, when the allele comprising the polymorphism is present Taq DNA polymerase adds nucleotides to the nonspecific primer until it reaches the specific primer. It then releases polynucleotides, the fluorescent dye and quenching agent from the specific primer through its endonuclease activity. The fluorescent dye is therefore no longer in proximity to the quenching agent and fluoresces. In the presence of the allele which does not comprise the polymorphism the mismatch between the specific primer and template inhibits the endonuclease activity of Taq and the fluorescent dye is not release from the quenching agent. Therefore by measuring the fluorescence emitted the presence or absence of the polymorphism can be determined.

In another method of detecting the polymorphism a polynucleotide comprising the polymorphic region is sequenced across the region which contains the polymorphism to determine the presence of the polymorphism.

Alternatively the presence of the polymorphism may be determined indirectly, for example by measuring an effect which the polymorphism causes. This effect may be in the expression or activity of the insulin receptor. Thus the presence of the polymorphism may be determined by measuring the activity or level of the expression of the insulin receptor in the individual.

The expression of the insulin receptor may be determined by directly measuring the level of the receptor in the cell or indirectly by measuring the level of any other suitable component in the cell, such as measuring mRNA levels (e.g. using quantitative PCR, such as by a Taqman based method).

In one embodiment the method is carried out in vivo, however typically it is carried out in vitro on a sample from the individual, typically a blood, saliva or hair root sample. The sample is typically processed before the method is carried out, for example DNA extraction may be carried out. The polynucleotide or protein in the sample may be cleaved either physically or chemically (e.g. using a suitable enzyme). In one embodiment the part of polynucleotide in the sample is copied (or amplified), e.g. by cloning or using a PCR based method. Polynucleotide produced in such a procedure is understood to be covered by the term “polynucleotide of the individual” herein.

Diagnostic Kit

The Example also provides a diagnostic kit that comprises a probe, primer, antibody (including an antibody fragment) or agent as defined herein. The kit may additionally comprise one or more other reagents or instruments (such as mentioned herein) which enable any of the embodiments of the method mentioned above to be carried out. Such reagents or instruments include one or more of the following: a means to detect the binding of the agent to the polymorphism, an enzyme able to act on a polynucleotide (typically a polymerase or restriction enzyme), suitable buffer(s) (aqueous solutions) for enzyme reagents, PCR primers which bind to regions flanking the polymorphism, a positive and/or negative control, a gel electrophoresis apparatus and a means to isolate DNA from sample.

Polynucleotides, Proteins and Antibodies

The Example further provides an isolated polynucleotide or protein that comprises (i) a polymorphism that causes susceptibility to cephalic pain or (ii) a naturally occurring polymorphism that is in linkage disequilibrium with (i). Such polymorphisms may be any of the polymorphisms mentioned herein. The polymorphism that causes susceptibility may be one which is or which is not found in nature.

The polynucleotide or protein may comprise human or animal sequence (or be homologous to such sequence). Such an animal is typically a mammal, such as a rodent (e.g a mouse, rat or hamster) or a primate. Such a polynucleotide or protein may comprise any of the human polymorphisms mentioned herein at the equivalent positions in the animal polynucleotide or protein sequence.

The polynucleotide or protein typically comprises the insulin receptor gene region sequence or the insulin receptor protein sequence, or is homologous to such sequences; or is part of (a fragment of) such sequences. Such sequences may be of a human or animal. In particular the part of the sequence may correspond to any of the sequences given herein in or parts of such sequences. The polynucleotide is typically at least 5, 10, 15, 20, 30, 50, 100, 200, 500, bases long, such as at least 1 kb, 10 kb, 100 kb, 1000 kb or more in length.

The polynucleotide is generally capable of hybridising selectively with a polynucleotide comprising all or part of the insulin receptor gene region sequence, including sequence 5′ to the coding sequence, coding sequence, intron sequence or sequence 3′ to the coding sequence. Thus it may be capable of selectively hybridising with all or part of the sequence shown in any one of SEQ ID NOS: 1 to 25 (including sequence complementary to that sequence).

Selective hybridisation means that generally the polynucleotide can hybridize to the gene region sequence at a level significantly above background. The signal level generated by the interaction between a polynucleotide of the invention and the gene region sequence is typically at least 10 fold, preferably at least 100 fold, as intense as interactions between other polynucleotides and the gene region sequence. The intensity of interaction may be measured, for example, by radiolabelling the polynucleotide, e.g. with ³²P. Selective hybridisation is typically achieved using conditions of medium to high stringency (for example 0.03M sodium chloride and 0.003 or 0.03M sodium citrate at from about 50° C. to about 60° C.).

Polynucleotides used in the method of the invention may comprise DNA or RNA. The polynucleotides may be polynucleotides which include within them synthetic or modified nucleotides. A number of different types of modification to polynucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art.

The protein used in the method of the invention can be encoded by a polynucleotide used in the method of the invention. The protein may comprise all or part of a polypeptide sequence encoded by any of the polynucleotides represented by SEQ ID NOS:1 to 25, or be a homologue of all or part of such a sequence. The protein may have one or more of the activities of the insulin receptor, such as being able to bind insulin and/or signalling activity. The protein is typically at least 10 amino acids long, such as at least 20, 50, 100, 300 or 500 amino acids long.

The protein may be used to produce antibodies specific to the polymorphism, such as those mentioned herein. This may be done for example by using the protein as an immunogen which is administered to a mammal (such as any of those mentioned herein), extracting B cells from the animal, selecting a B cell from the extracted cells based on the ability of the B cell to produce the antibody mentioned above, optionally immortalising the B cell and then obtaining the antibody from the selected B cell.

Polynucleotides or proteins used in the method of the invention may carry a revealing label. Labels are also mentioned above in relation to the method of the invention. Suitable labels include radioisotopes such as ³²P or ³⁵S, fluorescent labels, enzyme labels or other protein labels such as biotin.

Polynucleotides used in the method of the invention can be incorporated into a vector. Typically such a vector is a polynucleotide in which the sequence of the polynucleotide used in the method of the invention is present. The vector may be a recombinant replicable vector, which may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

The vector may be an expression vector. In such a vector the polynucleotide of the invention in the vector is typically operably linked to a control sequence which is capable of providing for the expression of the coding sequence by the host cell.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Such vectors may be transformed into a suitable host cell as described above to provide for expression of the protein of the invention. Thus, in a further aspect the invention provides a process for preparing the protein of the invention, which process comprises cultivating a host cell transformed or transfected with an expression vector as described above under conditions to provide for expression of the protein, and optionally recovering the expressed protein.

The vectors may be for example, plasmid, virus or phage vectors provided with an origin of replication, optionally a promoter for the expression of the said polynucleotide and optionally a regulator of the promoter. The vectors may contain one or more selectable marker genes. Promoters and other expression regulation signals may be selected to be compatible with the host cell for which the expression vector is designed.

The proteins and polynucleotides of the invention may be present in a substantially isolated form. They may be mixed with carriers or diluents which will not interfere with their intended use and still be regarded as substantially isolated. They may also be in a substantially purified form, in which case it will generally comprise at least 90%, e.g. at least 95%, 98% or 99%, of the dry mass of the preparation.

Homologs

Homologs of polynucleotide or protein sequences are referred to herein. Such homologs typically have at least 70% homology, preferably at least 80, 90%, 95%, 97% or 99% homology, for example over a region of at least 15, 20, 30, 100 more contiguous nucleotides or amino acids. The homology may be calculated on the basis of amino acid identity (sometimes referred to as “hard homology”).

For example the UWGCG Software Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff(1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differ by at least 1, 2, 5, 10, 20 or more mutations (which may be substitutions, deletions or insertions of nucleotide or amino acids). These mutation may be measured across any of the regions mentioned above in relation to calculating homology. In the case of proteins the substitutions are preferably conservative substitutions. These are defined according to the following Table. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Transgenic Animals

The method of the invention also can yield an animal transgenic for a polymorphism as mentioned above. The animal may be any suitable mammal such as a rodent (e.g. a mouse, rat or hamster) or primate. Typically the genome of all or some of the cells of the animal comprises a polynucleotide of the invention. Generally the animal expresses a protein of the invention Typically the animal suffers from cephalic pain and can be therefore used in a method to assess the efficacy of agents in relieving anti-cephalic pain. The transgenic model can further be used to assess the ability of agents to modulate insulin receptor signalling activity.

Treatment of Patients

The method of the Example provides a therapeutic method for treating a patient who has been diagnosed as being susceptible to cephalic pain by a method of the invention, comprising administering an effective amount of an anti-cephalic pain agent to the patient. The anti-cephalic pain agent may therefore be administered to a patient to prevent the onset of such pain or to combat an episode of cephalic pain. The method of the Exampl also provides:
use of an anti-cephalic pain agent in the manufacture of a medicament for use in treating a patient who has been diagnosed as being susceptible to cephalic pain by a method of the invention; and
a pharmaceutical pack comprising an anti-cephalic pain agent and instructions for administering of the agent to humans diagnosed by the method of the invention.

The anti-cephalic pain agent is typically an anti-migraine agent. Suitable anti-migraine agents are a steroid (e.g. hydrocortisone or dexamethasone, a NSAIDs (non-steroidal anti-inflammatory drug)(e.g. ibuprofen), a 5HT1D agonist, lidocaine (e.g. in the form of a nasal spray), an opioid (e.g. codeine or morphine), an Ergot preparation (e.g. ergotamine or dihydroergotamine), a triptan (e.g. sumatriptan, rizotriptan, naratriptan, zolmitriptan, eletriptan, frovatriptan or almotriptan), alniditan, metoclopramide, chlorpromazine, prochlorperazine, a beta-adrenergic antagonist (e.g. propranolol), a tricyclic antidepressant (e.g. amitriptyline), a calcium channel antagonists (e.g. verapamil or diltiazem), cyproheptadine, ALX-0646 (a trytamine analogue), LY334370, U109291, IS159 or PNU-142633.

An effective amount of such an agent may be given to a human patient in need thereof. The dose of agent may be determined according to various parameters, especially according to the substance used; the age, weight and condition of the patient to be treated; the route of administration; and the required regimen. A suitable dose may however be from 0.1 to 100 mg/kg body weight such as 1 to 40 mg/kg body weight. Again, a physician will be able to determine the required route of administration and dosage for any particular patient.

The formulation of the agent will depend upon factors such as the nature of the substance and the condition to be treated. Typically the agent is formulated for use with a pharmaceutically acceptable carrier or diluent. For example it may be formulated for oral, parenteral, intravenous, intramuscular or subcutaneous administration. A physician will be able to determine the required route of administration for each particular patient. The pharmaceutical carrier or diluent may be, for example, an isotonic solution.

The effectiveness of particular anti-cephalic agents may be affected by or dependent on whether the individual has particular polymorphisms in the insulin receptor gene region or insulin receptor. Thus the method of this Example allows the determination of whether an individual will respond to a particular anti-cephalic pain agent by determining whether the individual has a polymorphism which affects the effectiveness of that agent. There is further disclosed here a method of treating a patient who has been identified as being able to respond to the agent comprising administering the agent to the patient.

Similarly certain anti-cephalic pain agents may produce side effects in individuals with particular polymorphisms in the insulin gene region or protein. Thus the method of this Example can also allow the identification of a patient who is at increased risk of suffering side effects due to such an anti-cephalic agent by identifying whether an individual has such a polymorphism.

Individuals who carry a particular polymorphism in the insulin receptor gene may exhibit differences in their ability to regulate metabolic pathways under different physiological conditions and will display altered reactions to different diseases. In addition, differences in metabolic regulation arising as a result of the polymorphism may have a direct effect on the response of an individual to gene therapy. The polymorphism may therefore have the greatest effect on the efficacy of drugs designed to modulate the activity of the insulin receptor or other components in its signalling pathway. However, the polymorphisms may also affect the response to agents acting on other biochemical pathways regulated by the insulin receptor. The invention may therefore be useful both to predict the clinical response to such agents and to determine therapeutic dose.

In a further aspect, the invention can be used to assess the predisposition and /or susceptibility of an individual to diseases mediated by the target gene found, in this case, the insulin receptor. Polymorphism may be particularly relevant to the development of such diseases. The present invention may be used to recognise individuals who are particularly at risk from developing these conditions.

In a further aspect, the method of the invention exemplified here may further be used in the development of new drug therapies which selectively target one or more allelic variants of the insulin receptor gene (i.e. which have different polymorphisms). Identification of a link between a particular allelic variant and predisposition to disease development or response to drug therapy may have a significant impact on the design of new drugs. Drugs may be designed to regulate the biological activity of the variants implicated in the disease process while minimising effects on other variants.

The following Examples illustrates the invention:

### EXAMPLE 1-A

### Association Study

Clinical Criteria for Identifying Individuals with Migraine

The following criteria were used to identify individuals with specific types of migraine:
Migraine without aura:
   HA (head ache) lasting 4-72 hrs if unsuccessfully treated;
   HA with at least 2 of the following: unilateral pain; pulsating quality; moderate to severe intensity; aggravation by physical activity;
   HA with nausea, or vomiting, or photophobia, or phonophobia (at least 1).
Migraine with aura:
   Aura lasting 4-60 minutes;
   HA defined as above, with onset accompanying or following aura within 60 minutes.
Familial hemiplegic migraine:
   HA fulfills migraine with aura characteristics;
   aura includes hemiparesis that may be prolonged (>60 minutes): at least 1 first-degree relative with similar HAs.
      Genotyping of Individuals for SNPs

Samples were obtained from the study group and genomic DNA extracted using a standard kit and a slating out technique (Cambridge Molecular). The genotypes of the migraineurs with aura and control individuals for individual SNPs within the insulin receptor gene were then determined from the DNA samples obtained using the Taqman allelic discrimination assay.

For each polymorphic site the allelic discrimination assay used two allele specific primers labeled with a different fluorescent dye at their 5′ ends but with a common quenching agent at their 3′ ends. Both primers had a 3′ phosphate group so that Taq polymerase could not add nucleotides to them. The allele specific primers comprised the sequence encompassing the polymorphic site and differed only in the sequence at this site. The allele specific primers were only capable of hybridizing without mismatches to the appropriate allele.

The allele specific primers were used in typing PCRs in conjunction with a third primer, which hybridized to the template 5′ of the two specific primers. If the allele corresponding to one of the specific primers was present the specific primer would hybridize perfectly to the template. The Taq polymerase, extending the 5′ primer, would then remove the nucleotides from the specific probe releasing both the fluorescent dye and the quenching agent. This resulted in an increase in the fluorescence from the dye no longer in close proximity to the quenching agent.

If the allele specific primer hybridized to the other allele the mismatch at the polymorphic site would inhibit the 5′ to 3′ endonuclease activity of Taq and hence prevent release of the fluorescent dye.

The ABI7700 sequence detection system was used to measure the increase in fluorescence from each specific dye during the thermal cycling PCR directly in PCR reaction tubes. The information from the reactions was then analyzed. If an individual was homozygous for a particular allele only fluorescence corresponding to the dye from that specific primer would be released, if the individual was heterozygous both dyes would fluoresce.

Table 1 shows the SNPs typed in the sample group to determine association of the SNP with migraine. The polymorphic site typed is given together with the flanking sequence 5′ and 3′.

TABLE 1

| Associated SNP | P value is association study |
|---|---|
| INSB | 0.002 |
| INSC | 0.007 |
| X8po11 | 0.018 |
| X11po11 | 0.05 |
| Xl7po12 | 0.008 |

Table 2 shows the P values for the co-inheritance of the associated SNPs with migraine.

### EXAMPLE 2

### Functional Effect of Polymorphisms in the Insulin Receptor

60 female subjects with migraine were divided into 2 groups: first, a group of 21 who had one or more SNP-associated alleles with the following SNPS: INSC, INSB, and exon17; and second, a group of 39 who had none of these SNP-associated alleles (i.e. wild-type alleles at these sites). Polymorphism typing was performed using the Taqman assay described in Example 1. A radioligand binding assay (based on the assay described in Kotterman et al (1981) J. Clin. Invest. 68, 957-69) was used to measure the binding of insulin to the insulin receptor of subjects in the two groups. The group with the SNP-associated alleles had significantly reduced INSR radioligand binding (0.042+/−0.005 fmole insulin bound per million monocytes) compared to the group with wild-type alleles (0.056+/−0.004 fmole insulin bound per million monocytes; p=0.03). This finding demonstrates that SNP-associated alleles of INSR confer significantly reduced INSR radioligand binding compared to wild-type alleles, suggesting that insulin sensitising agents may be used to treat patients with cephalic pain.

The above described methods are iteratively and/or in parallel applied to multiple disease states found in individuall members of the patient populations that have been enrolled in the clinical trial. In this fashion, the population then yields information about associations across a wide spectrum of diseases, thereby reducing the cost of biomedical research in the pursuit of information about associations between genotypes and disease phenotypes. Such association information is then used to forecast what points of intervention are most likely to be worthwhile targets for pharmacologic intervention in the treatment of human disease, as an overall aid to the discovery of novel human therapeutic drugs.

1. A method of datamining data obtained from a population of humans in clinical trials, across multiple diseases, for associations between said diseases and multiple genotypes, and performed in a programmable digital computer, comprising the steps of:
(a). providing a database having, for each member of a subject population, a first value set specifying at least one polymorphic form selected from a plurality of polymorphic forms present in said population at at least one genetic locus exhibiting polymorphism, and a second value set specifying a plurality of phenotypes, wherein at least one of said polymorphic forms is not known to have a statistically significant correlation with at least one of said phenotypes; and
(b). determining all possible statistical correlations between the plurality of polymorphic forms and the plurality of phenotypes.
